(19) United States
(12) Patent Application Publication
(10) Pub. No.: US 2021/0074384 A1
(43) Pub. Date: Mar. 11, 2021

(54) METHOD AND SYSTEM FOR CHARACTERIZATION OF METABOLISM-ASSOCIATED CONDITIONS, INCLUDING DIAGNOSTICS AND THERAPIES, BASED ON BIOINFORMATICS APPROACH

(71) Applicant: PSOMAGEN, INC., Rockville, MD (US)

(72) Inventors: Zachary Apte, San Francisco, CA (US); Jessica Richman, San Francisco, CA (US); Daniel Almonacid, San Francisco, CA (US); Inti Pedroso, San Francisco, CA (US); Victoria Dumas, San Francisco, CA (US); Valeria Marquez, San Francisco, CA (US); Ingrid Araya, San Francisco, CA (US); Ricardo Castro, San Francisco, CA (US); Mario Saavedra, San Francisco, CA (US); Melissa Alegria, San Francisco, CA (US)

(21) Appl. No.: 16/755,233

(22) PCT Filed: Mar. 18, 2019
(86) PCT No.: PCT/US2019/022807

§ 371 Date: Apr. 10, 2020

Related U.S. Application Data
(60) Provisional application No. 62/644,347, filed on Mar. 16, 2018.
(60) Provisional application No. 62/679,783, filed on Jun. 2, 2018.
(60) Provisional application No. 62/679,785, filed on Jun. 2, 2018.
(60) Provisional application No. 62/679,787, filed on Jun. 2, 2018.
(60) Provisional application No. 62/724,928, filed on Aug. 30, 2018.
(60) Provisional application No. 62/737,108, filed on Sep. 27, 2018.
(60) Provisional application No. 62/759,975, filed on Nov. 12, 2018.
(60) Provisional application No. 62/770,919, filed on Nov. 23, 2018.

(51) Int. Cl. G16B 40/20; A61B 5/00; G16B 40/30; G16B 20/00; G16B 15/00; G16H 50/20; G16H 50/30; G16H 70/40; G16H 20/10; G16H 50/50; G16H 20/60

(52) U.S. Cl. CPC G16B 40/20; G16H 10/60; A61B 5/4866; A61B 5/4848; A61B 5/4845; A61B 5/486; G16B 40/30; G16B 20/00; G16B 15/00; G16H 50/20; G16H 50/30; G16H 70/40; G16H 20/10; G16H 50/50; G16H 20/60; A61B 5/7264

(57) ABSTRACT

Embodiments of a method and/or system (e.g, for metabolism-related prediction) can include: generating an enzyme dataset; generating a substrate dataset; generating a metabolism model such as for predicting an enzyme feature associated with metabolism of a query molecule, based on the enzyme dataset and/or the substrate dataset; determining a microorganism taxon (and/or microorganism taxa) S140 associated with the metabolism of the query molecule based on one or more predicted enzyme features of the metabolism model (e.g., machine learning model; etc.) and/or determining a query molecule score (e.g., drug score) for one or more users based on the microorganism taxon.

### CROSS-REFERENCE TO RELATED APPLICATIONS

This application claims the benefit of U.S. Provisional Application Ser. No. 62/644,347 filed 16 Mar. 2018, U.S. Provisional Application Ser. No. 62/679,783 filed 2 Jun. 2018, U.S. Provisional Application Ser. No. 62/679,785 filed 2 Jun. 2018, U.S. Provisional Application Ser. No. 62/679,787 filed 2 Jun. 2018, U.S. Provisional Application Ser. No. 62/724,928 filed 30 Aug. 2018, U.S. Provisional Application Ser. No. 62/737,108 filed 27 Sep. 2018, U.S. Provisional Application Ser. No. 62/759,975 filed 12 Nov. 2018, U.S. Provisional Application Ser. No. 62/770,919 filed 23 Nov. 2018, which are each incorporated in its entirety herein by this reference.

### TECHNICAL FIELD

The disclosure generally relates to microorganism-associated metabolism.

### BACKGROUND

There are 1:1 microbial cells in the human cells of our body, most of which reside in the intestine (100 trillion cells and 5 million unique genes). Specifically, the most relevant phyla that are found in the human intestine are: Firmicutes, Bacteroidetes, Actinobacteria, Proteobacteria, Fusobacteria and Verrucomicrobia. The intestinal microbiota is involved in many aspects such as the production of vitamins and metabolites, the metabolism of drugs, protection against pathogens and the modulation of the immune system, among others; and it can be highlighted that the factors that modulate the gut microbiota are: lifestyle, immune system, previous infections and medical surgery, use of medications, among others.

Humans ingest a large number of foreign small molecules denominated xenobiotics. In this group, we can find dietary components, environmental chemicals, and pharmaceuticals. The trillions of microorganisms that inhabit our gastrointestinal tract can directly alter the chemical structures of xenobiotics compounds. Gut microbes modify many classes of dietary compounds, including polysaccharides, lipids, proteins, and phytochemicals complexes. These metabolic reactions are linked to health benefits, different conditions, as well as diseases. In a specific manner, gut microbial xenobiotic metabolites are known to have altered bioactivity, bioavailability, and toxicity, and can interfere with the activities of human xenobiotic-metabolizing enzymes to affect the fates of other ingested molecules. Whereby, bacteria and enzymes will provide both specific targets for manipulation and diagnostic markers that can be incorporated into clinical studies and practice. But, in the majority of cases, the individual microbes and enzymes that mediate these reactions are unknown.

Xenobiotics compounds can encounter gut microbes via multiples routes, for example orally ingested compounds pass the upper gastrointestinal tract to the small intestine where they can be modified by gut enzymes and absorbed by host tissues. They can also reach liver by the portal vein. Meanwhile, intravenously administered compounds can be introduced to systemic circulation. Then, they can be further metabolized or excreted via the biliary duct back to the gut lumen or through the kidneys; and if the metabolites reach the gut lumen, they can continue to the large intestine to be eventually excreted.

Another important issue is how the microbiome modifies dietary compounds, environmental chemicals, and pharmaceuticals. The transformations performed by gut microbial can be by hydrolytic transformations -by hydrolase enzymes (proteases, glycosidases and sulfatases) that catalyze the addition of water molecules to a substrate, followed by bond cleavage-, lyase reductions -by lyase enzymes breaking C—C or C—X bonds (where X=O, N, S, P or halides) without relying on oxidation or addition of water-, reductive transformations -by reductase enzymes that reduce a wide range of functional groups, including alkenes and α,β-unsaturated carboxylic acid derivatives, nitro-, N-oxide, azo-, and sulfoxide groups using various cofactors (NAD(P)H, flavin, Fe—S clusters, etc.) to mediate the transfer of electrons or hydride equivalents to substrates-, functional group transfer reactions -by transferase enzymes that move functional groups (for example methyl and acyl groups) between two substrates using nucleophilic substitution reactions-, and transformations mediated by radical enzymes. -predominating the anaerobic metabolism, where enzymes typically generate a substrate-based radical intermediate through single-electron transfer or homolytic bond cleavage. This initial substrate-based radical is then converted to a product-based radical. Formation of the final product often regenerates the initial enzyme- or cofactor-based radical to complete the catalytic cycle-.

As mentioned above, xenobiotics molecules can be derived by many sources, as for example dietary components. In this matter, some specific examples of this components are gluten -found in wheat-based food-, cholesterol -found in meats, fish, eggs, cheese, etc.-, alcohol -found in alcoholic drinks-, choline -found for example in meat-, among others. In this matter, it is important to know what is doing the gut microbiota with the food -and the components of this- that we eat.

In the case of gluten, there is an autoimmune disorder called celiac disease, characterized by an inflammatory response to dietary gluten in wheat-based food. Small intestinal microbes from patients with celiac disease interact with gluten trigger a different immune reaction than the microbes from a person without celiac disease. In comparative studies of stool samples from patients with and without celiac disease, it has been seen that in stool samples from patients with celiac disease has been detected the bacteria Pseudomonas aeruginosa, that it correlates with the production of highly immunogenic peptides by altering gluten proteolysis; meanwhile, in stool samples from patients without celiac disease has been detected Lactobacillus sp., bacteria that can degrade peptides to decrease immune reaction.

In another example, ingested cholesterol can be absorbed in the small intestine and undergo biliary excretion and enterohepatic circulation. Has been reported that gut microbes -as Eubacterium coprostanoligenes by an enzyme not yet identified- can reduce cholesterol generating coprostanol, which cannot be reabsorbed and is excreted, removing cholesterol from circulation.

In the case of alcohol that we drink, gut microbiota have alcohol dehydrogenase enzymes capable of breaking down alcohol and convert it to acetaldehyde. The accumulation of acetaldehyde has toxic properties, which are associated to several conditions, from hangover symptoms to colon pathologies including cancer. Besides, high acetaldehyde levels can cleave folate to inactive forms via acetaldehyde/xanthine oxidase-generated superoxide; and folate deficiency has been associated to increased risk of colonic cancer.

Respect with meat and foods rich in choline -such as poultry, fish, dairy products, pasta, rice, etc.-, that have phosphatidylcholine, the intestinal microbes form Trimethylamine (TMA) of this molecule and then the flavin hepatic monooxygenase (FMO) of the host catalyze the conversion of TMA into trimethylamine N-oxide (TMAO), which enhances atherosclerosis in animal models and is associated with cardiovascular risks in clinical studies. Another way to generate TMA, and subsequent TMAO, in mammals is through dietary intake of L-carnitine from carnitine-containing food -as meat-, where a significant proportion of this dietary carnitine can be further metabolized by microbiota before absorption, generating TMA, which is oxidized to TMAO by hepatic FMO, and increased the risk of atherosclerosis and cardiovascular risk. In a specific manner, exists to main pathways for bacterial TMA production: by microbial choline TMA lyase using choline as a substrate and by carnitine-to-TMA enzyme using L-carnitine as a substrate. In the first mentioned, the microbial choline TMA lyase has been reported as a unique glycyl radical employing enzyme complex comprised of a catalytic polypeptide, CutC, and an associated activating protein, CutD, encoded by adjacent genes within a gene cluster; meanwhile in the second enzyme mentioned is composed of an oxygenase component (CntA) and a reductase component (CntB), where CntA belong to a uncharacterized group of Rieske-type proteins, which are best known for ring-hydroxylation of aromatic hydrocarbons.

Other important source of xenobiotic are pharmaceuticals, and it is important to know in which manner the microbiome modifies or competes or interferes with drugs. In a specific example, the acetaminophen (or paracetamol) is metabolized in the liver producing two types of inactive metabolites: acetaminophen sulfate and acetaminophen glucuronide, and also a toxic one: N-acetyl-p-benzoquinone imine (NAPQI). A microbial metabolite, p-cresol sulfate, was found to be inversely associated with the ratio of acetaminophen sulfate to acetaminophen glucuronide. p-cresol is produced by several bacteria: Firmicutes (Clostridium difficile), Bacteroidetes, Actinobacteria and Fusobacteria phyla. Notably, p-cresol is metabolized in the liver to p-cresol sulfate, and both p-cresol and acetaminophen are substrates of the human cytosolic sulfotransferase 1A1 (SULTA1), so that competition between p-cresol and acetaminophen impedes detoxify acetaminophen, increasing the accumulation of NAPQI, causing subsequent liver damage.

In another example microbial metabolism can also interfere with the bioavailability of drugs, as digoxin. Digoxin is a drug for congestive heart failure extracted from Digitalis purpurea, and has a very narrow therapeutic window, requiring careful monitoring to avoid toxicity. In this sense, over 10% of patients treated with digoxin excrete high levels of dihydrodigoxin, an inactive metabolite derived from reduction of an α,β-unsaturated lactone. Subsequent studies and isolations, revealed a digoxin-metabolizing microbe, Eggerthella lenta, responsible of reductive metabolism leads to digoxin inactivation. In a specific manner, E. lenta has a cardiac glycoside reductase (cgr) operon that encodes two proteins that resemble bacterial reductases involved in anaerobic respiration: a membrane-associated cytochrome (Cgr1) transfers electrons through a series of hemes to a flavin-dependent reductase (Cgr2) that converts digoxin to dihydrodigoxin.

Also exists the case of bacterial reactivation of drugs, like irinotecan. Irinotecan (CPT-11) is a prodrug of SN-₃8 (a topoisomerase inhibitor used for treating cancer). SN-₃8 is activated by host carboxylesterases. SN-₃8 is glucuronidated by host liver enzymes into an inactive compound (which reaches the gut by biliary excretion). Bacterial beta-glucuronidases can reactivate SN-₃8 in the large intestine, provoking toxicity by SN-₃8 overdosing, causing intestinal damage and diarrhea in cancer patients. Thus, generating beta-glucuronidases inhibitors to avoid secondary effects of reactivation of Irinotecan is attractive. As these enzymes are broadly distributed in commensal bacteria and are present in humans, inhibitors need to be selective for bacterial β-glucuronidases and non-toxic to both host cells and other gut microbes. Some investigations showed that selectivity of potential inhibitors is based on a loop unique to bacterial β-glucuronidases, so the inhibitors based on this approximation were highly effective against the enzyme target in living aerobic and anaerobic bacteria, but did not kill the bacteria or harm mammalian cells; besides oral administration of this inhibitors protected mice from irinotecan-induced toxicity. Other case related to bacterial reactivation is for non-steroidal anti-inflammatory drugs (NSAIDs). These drugs are used to reduce pain that is associated with inflammation -as like premenstrual cramping or chronic inflammation in the case of arthritis-. Besides that, NSAIDs have been the cause of 43% of drug-related emergency visits in the United States. Extended use of NSAIDs can cause ulcers or irritate lining of digestive tract. Some examples of NSAIDs are diclofenac, ibuprofen, aspirin, diflunisal, etc. NSAIDs are processed to its glucuronide metabolite by UDP-glucuronosyltransferase (UGT) enzymes. Diclofenac-glucuronide is reactivated in the second half of the small intestines by β-glucuronidase enzymes expressed by the symbiotic microbiota.

### BRIEF DESCRIPTION OF THE FIGURES

FIG. 1 includes a specific example of drug score prediction.

FIG. 2 includes a specific example of a drug metabolism predictor, where bacteria associated with Omeprazole metabolism were identified.

FIGS. 3A-3E includes specific examples of a 5-step process associated with metabolism prediction, where each of the steps can be performed in any suitable order at any suitable time and frequency.

FIG. 4 includes a specific example of an artificial sweetener-related recommendation.

FIG. 5 includes a specific example of an alcohol-related recommendation.

FIG. 6 includes a specific example of an alcohol-related recommendation.

FIG. 7 includes a specific example of an alcohol-related recommendation.

FIG. 8 includes a specific example of an alcohol-related recommendation.

FIGS. 9A-9F includes specific examples alcohol metabolism-related recommendations.

FIG. 10 includes a specific example of an alcohol metabolism-related recommendation.

### DESCRIPTION OF THE EMBODIMENTS

The following description of the embodiments (e.g., including variations of embodiments, examples of embodiments, specific examples of embodiments, other suitable variants, etc.) is not intended to be limited to these embodiments, but rather to enable any person skilled in the art to make and use.

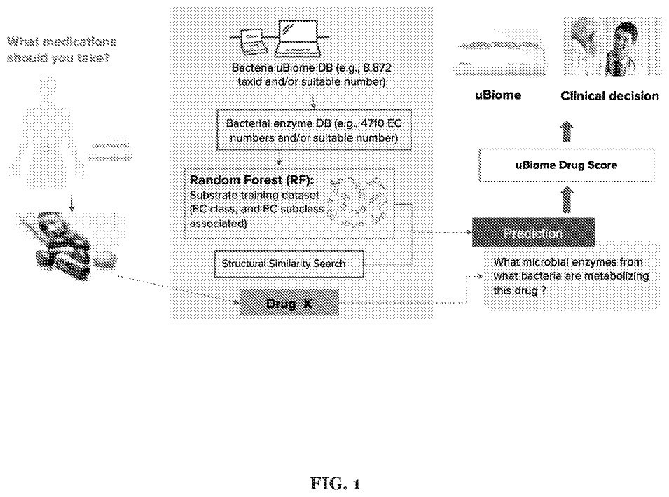

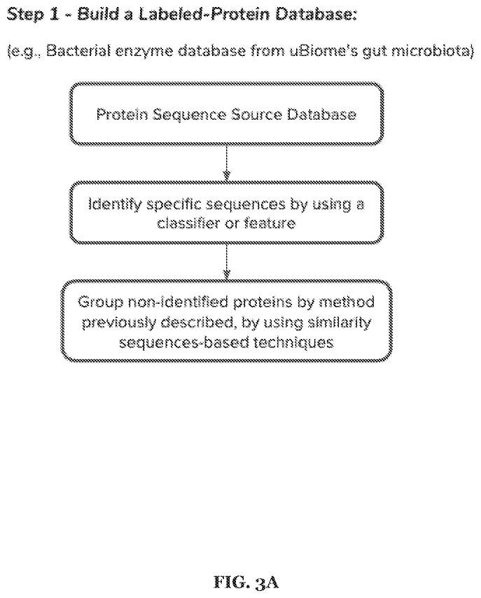

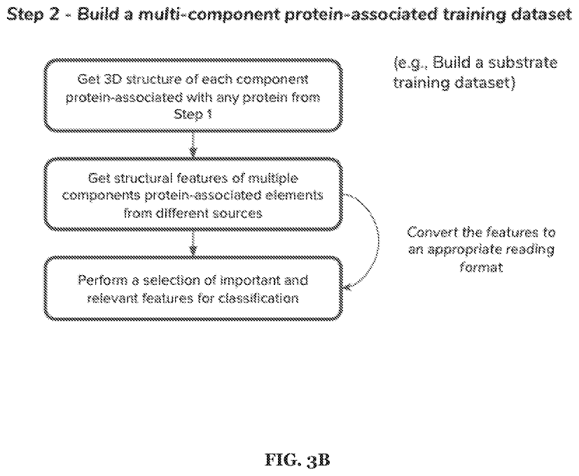

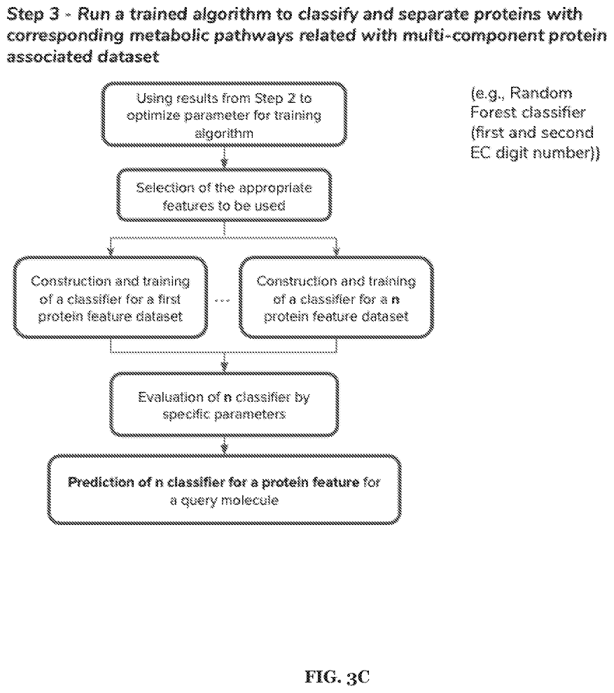

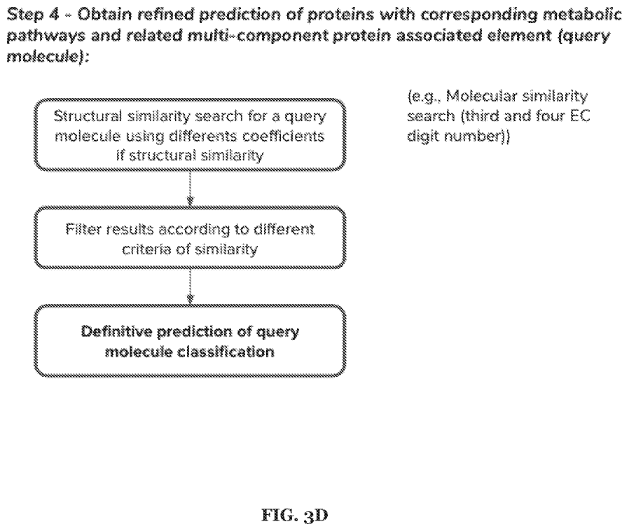

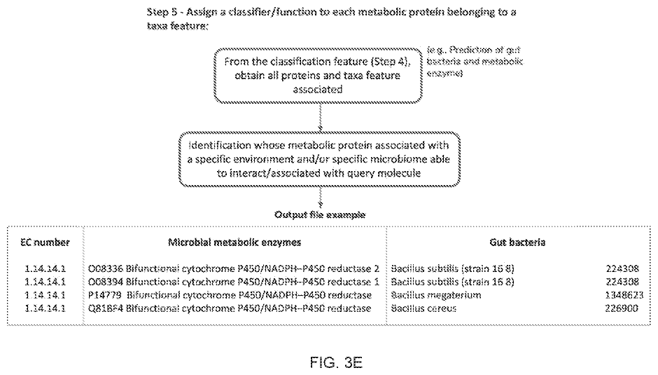

Embodiments of a method 100 (e.g., for metabolism-related prediction; specific example as shown in FIGS. 3A-3E) can include: generating an enzyme dataset Silo; generating a substrate dataset S120; generating a metabolism model (e.g., machine learning model; etc.) S130, such as for predicting an feature (e.g., Enzyme Commission number feature, such as class number, sub-class number, sub-sub class number, sub-sub-sub class number, etc.), associated with metabolism of a query molecule, based on the enzyme dataset and/or the substrate dataset; determining a microorganism taxon (and/or microorganism taxa) S140 associated with the metabolism of the query molecule based on one or more predicted enzyme feature outputs of the metabolism model (e.g., machine learning model; etc.) and/or determining a query molecule score (e.g., drug score) for one or more users based on the microorganism taxon (and/or microorganism taxa) and/or a microbiome characterization (e.g., indicating microorganism composition and/or microorganism function, such as microorganism composition diversity and/or microorganism functional diversity; etc.) for the user, where the query molecule score is associated with the query molecule (e.g., a drug score indicating drug efficacy such as in relation to drug metabolism for the drug for the user; such as shown in FIG. 1 etc.).

Additionally or alternatively, the method 100 can include promoting (e.g., providing; administering; recommending; presenting; etc.) a therapy to the user for a microorganism-related condition based on the drug score (and/or any suitable model outputs and/or suitable data described herein; etc.). In a specific example, promoting (e.g., providing, etc.) a therapy can include providing one or more recommendations for one or more therapies to the user. Additionally or alternatively, the method 100 can include performing a structural similarity search to filter the plurality of enzymes, based on the substrate dataset and query molecule structural features of the query molecule.

Enzyme datasets can include enzyme data (e.g., enzyme data indicating a set of enzymes associated with a set of microorganism taxa; etc.), chemical reaction data (e.g., Enzyme Commission (EC) numbers for the set of enzymes indicated by the enzyme data; etc.) associated with the set of enzymes; and/or any suitable data related with enzymes. In a specific example, the chemical reaction data includes Enzyme Commission number data associated with the set of enzymes. In variations, the method 100 can include annotating enzymes without an associated Enzyme Commission number, such as based on enzymes with associated Enzyme Commission number data. In a specific example, the set of enzymes includes a first subset of enzymes unassociated with the Enzyme Commission number data and a second subset of enzymes associated with the Enzyme Commission number data, and where generating the enzyme dataset includes annotating the first subset of enzymes based on the Enzyme Commission number data.

Substrate datasets can include substrate structural features associated with a set of substrates (e.g., substrates actable upon by the set of enzymes; etc.). Substrate structural features can include any one or more of: 3D structural features associated with the set of substrates; product molecule features (e.g., data indicating the products produced from the one or more enzymes reacting with the one or more substrates; etc.); drug features (e.g. interactions between the enzymes, substrates, and one or more drugs; types of drugs affected by the processes associated with the enzymes and/or substrates; etc.) associated with the set of substrates; and/or any suitable features associated with substrates. In a specific example, the method 100 can include, for each substrate of the set of substrates, identifying a subset of relevant features (e.g., through any suitable feature selection algorithm and/or approach; etc.) from the 3D structural features, the product molecule features, and/or the drug features, and/or where generating the machine learning model includes generating the machine learning model for predicting the enzyme associated with metabolism of the query molecule based on the enzyme dataset and the subset of relevant features.

In variations, the method 100 can additionally or alternatively include predicting an Enzyme Commission class number and/or an Enzyme Commission sub-class number for the query molecule, based on the predicted enzyme output and/or any suitable data, and/or where determining the microorganism taxon includes determining the microorganism taxon based on the Enzyme Commission class number and/or an Enzyme Commission sub-class number.

The metabolism model, suitable portions of embodiments of the method 100, suitable portions of embodiments of the system 200, can include, apply, employ, perform, use, be based on, and/or otherwise be associated with artificial intelligence approaches (e.g., machine learning approaches, etc.) including any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable artificial intelligence approach. In a specific example, the machine learning model includes a random forest model for predicting the enzyme, of the set of enzymes, associated with metabolism of the query molecule. In a specific example, generating the machine learning model includes generating the machine learning model for predicting a plurality of enzymes, of the set of enzymes, associated with the metabolism of the query molecule. In a specific example, the method 100 can further include determining a plurality of microorganism taxa including the microorganism taxon associated with the metabolism of the query molecule based on a set of predicted enzyme outputs including the predicted enzyme output of the machine learning model, where the set of predicted enzyme outputs indicate the plurality of enzymes.

In a specific example, the chemical reaction data includes Enzyme Commission number data associated with the set of enzymes, and where the enzyme feature includes at least one of an EC class number and an EC sub-class number for the query molecule (and/or EC sub-sub class number, EC sub-sub-sub class number, any suitable EC-related feature; etc.). In a specific example, the Enzyme Commission number feature includes an Enzyme Commission class number and an Enzyme Commission sub-class number for the query molecule, wherein the method can additionally or alternatively include predicting an Enzyme Commission sub-sub-class number and/or an Enzyme Commission sub-sub-sub-class number for the query molecule, such as based on similarity (e.g., using any suitable coefficients of similarity, etc.) between query molecule structural features and the substrate structural features, and/or wherein determining the microorganism taxon can include determining the microorganism taxon based on the Enzyme Commission class number, the Enzyme Commission sub-class number, the Enzyme Commission sub-sub-class number, and the Enzyme Commission sub-sub-sub-class number.

In a specific example, predicted microorganism taxa are associated with a human gut microbiome, but any suitable metabolism model outputs and/or any identified microorganism taxa can be associated with any suitable body sites including any one or more of: gut, skin, nose, mouth, genitals (e.g., vagina, etc.) and/or other suitable body sites.

In a specific example, determining the microbiome characterization can based on a microorganism composition diversity dataset and/or a microorganism functional diversity dataset for the user.

In a specific example, the query molecule includes at least one of a vitamin-related molecule, an artificial sweetener-related molecule, and an alcohol-related molecule.

Embodiments of a system 200 and/or platform (e.g. for metabolism-related prediction) can include: a first module for capturing data (e.g., survey, literature, user metadata, sample analysis, bacteria databases; etc.), a second module including a metabolism predictor tool able to identify any single molecule derived from any gut microbiota (e.g. enzymes, metabolites, compounds) that can metabolize a query molecule (e.g. drug, metabolite), such as using machine learning techniques and chemoinformatics, a third module for of determination of microorganism taxa associated with metabolism of query molecules, a fourth module for personalized dietary recommendations, a fifth module for precision medicine, a sixth module for informing toxicology risk assessment, a seventh module for improving drug discovery and drug development, an intermediate outcome that is a prediction which enters to the fourth, fifth, sixth and seventh modules for the prediction processing in each of them, and/or a final and independent outcome -such coming from the modules- including any molecule that are potential drugs, metabolites, therapeutic agent, etc. related or not to a condition, and/or for other suitable purposes.

The first module for capturing data can include: any mechanism, technique, method or suitable methodology to capture data related or not to a condition, such as including one or more of survey data, literature, user metadata, sample analysis, bacteria databases (e.g., including associations between microorganism taxa and microorganism-related conditions, etc.), among others.

The second module can include a metabolism predictor tool including: a methodology to build a molecule (e.g. peptide) predictor that can be described in a specific example as follows: first build a protein database, by identifying a group of species of interest (e.g. bacteria from the microbiota, microorganisms in any sample). Then, obtain reference proteomes for each species and annotate (e.g. classify) those protein that do not have a proper protein feature associated (using, e.g. BLAST, sequence similarity networks (SSN), Clustal, HMMs or any other sequence similarity search algorithm). Second, build a substrate database, where substrates are associated with each protein feature and are obtained in tridimensional format and later converted to a structural features (e.g. fingerprints, ADME properties, chemical and biological descriptors, and many others). Structural features format allows to properly describe the structural features of the molecule in a numerical form. Third, a machine learning classification method (e.g. random forest, support vector machine, decision trees, neural networks, Naïve Bayes, AdaBoost, Bagging, IBk, MultiClass classifier, etc.) is performed to predict the protein feature in relation to a query molecule. Fourth, a structural similarity search (using e.g. Tanimoto coefficient, Tversky coefficient, or Dice similarity coefficient) is carried out to get more protein features. Then, as a final result, the metabolic protein and the corresponding species involved in the protein feature in relation to a query molecule will be identified. However, any suitable processes can be applied in any suitable order for facilitating determination of a protein feature predictor tool.

The third module can include determination of microorganism taxa associated with metabolism of a query molecule.

The fourth module for personalized dietary recommendations, can include: deliver nutritional intervention, advice, guidance, services or products suited to each individual to preserve or increase their health.

The fifth module for precision medicine, can include: take into account individual variability in genes, environment, lifestyle, etc., of each person to the treatment and prevention for a particular condition.

The sixth module for informing toxicology risk assessment, can include: process or method that considers toxicological hazard and risk identification, toxicological risk analysis, toxicological risk evaluation and toxicological risk control; with the aim to remove or minimize the toxicological risk or adverse effects on individuals. The toxicological risk can consider chemicals, physical agents, pharmaceuticals, biological agents, among others.

The seventh module for improve drug discovery and drug development, can include: upgrade, refine, enhance target discovery, target selection, identification of potential lead compounds, lead optimization, development phase (preclinical stage), proof of concept, development, product differentiation, registration and launch of a novel drug or therapeutic agent.

The intermediate outcome that is a prediction, can include: forecast or predictions based on data described herein.

A final and independent outcome, can include: any molecule that are potential drugs, metabolites, therapeutic agent, supplement, dietary compounds, formulations, etc. related or not to a condition, and/or for other suitable purposes.

Embodiments of the system can function for prediction of a protein function based on a protein feature in association with any multi-component protein-associated element (e.g. a query molecule). The use of currently disclosed embodiment of a system, for metabolism of any query molecule, where a query molecule can include: drugs, other classes of xenobiotics (e.g. dietary compounds, environmental chemicals) and any other multi-component protein-associated, element.

Embodiments of the system (e.g., for metabolism-related prediction) can include a data collection module for collecting (and/or a protein-related database including): protein data indicating a set of proteins associated with a set of microorganism taxa, chemical reaction data associated with the set of proteins, and/or substrate data comprising substrate structural features associated with a set of substrates associated with the set of proteins and/or other suitable data described herein; a metabolism module (e.g., metabolism machine learning model) for predicting a protein feature (e.g., EC number feature) associated with metabolism of a query molecule, based on the protein data, the chemical reaction data, and/or the substrate data; and/or a microorganism module for determining a microorganism taxon associated with the metabolism of the query molecule based on the protein feature predicted from the metabolism module for the query molecule.

In variations, the system can additionally or alternatively include a drug score module for predicting a drug score indicating a drug efficacy for a user for the query molecule based on the microorganism taxon and a microbiome characterization for the user. In variations, the system can additionally or alternatively include a microbiome characterization module for determining the microbiome characterization based on a microorganism composition diversity dataset and a microorganism functional diversity dataset for the user. In variations, the system can additionally or alternatively include a therapy module for determining a therapy for the user based on the drug score. In variations, the system can additionally or alternatively include a therapy provision module for providing the therapy to the user. In variations, the system can additionally or alternatively include a personalized dietary recommendation module for determining a personalized dietary recommendation for a user based on a microbiome characterization for the user and the microorganism taxon associated with the metabolism of the query molecule, and/or wherein the personalized dietary recommendation comprises at least one of a vitamin-related recommendation, an artificial sweetener-related recommendation, and/or an alcohol-related recommendation. In a specific example, the personalized dietary recommendation includes the alcohol-related recommendation associated with a set of microorganism taxa comprising at least one of: Bacteroides uniformis (species); Holdemania filiformis (species); Turicibacter sanguinis (species); Eisenbergiella tayi (species); Erysipelatoclostridium ramosum (species); Dielma fastidiosa (species); Roseburia hominis (species); Catenibacterium mitsuokai (species); Solobacterium moorei (species); Eggerthia catenaformis (species); Allobaculum stercoricanis (species); and/or Lactobacillus (genus).

In specific examples Metabolism predictor can be used for drug metabolism, but its use can be expanded to predict the metabolism of other classes of xenobiotics, such as dietary compounds, environmental chemicals, etc.

In specific examples, as a drug metabolism predictor, bacteria associated to Omeprazole metabolism were also identified, as shown in FIG. 1. Omeprazole is a medication used in the treatment of gastroesophageal reflux disease. As an example, the distribution of bacteria that metabolize omeprazole was obtained in stool samples. An example of use for that information is the generation of a “score” based on the sum of relative abundances of taxa identified with metabolism predictor. Such a score will allow to inform users about their ability to metabolize a drug, or the propensity that a drug does not have the expected effects. Then, gaining a better understanding of the specific organisms and enzymes responsible for these activities and their presence in patients could aid in drug selection and dosing.

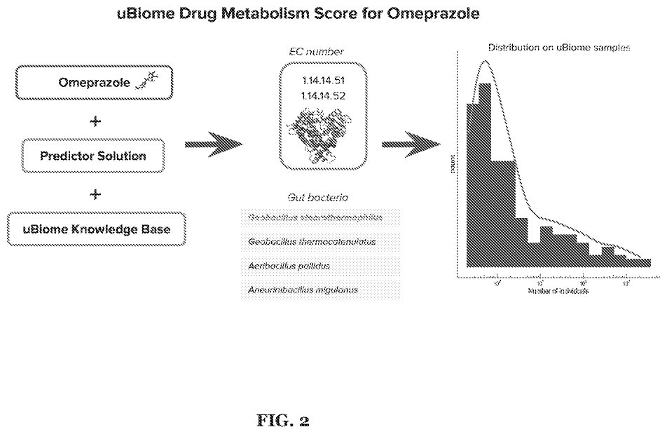

In a specific examples, the embodiment of the methodology described previously was applied in the following example, where the proteins are enzymes and the protein feature can include the EC Number. The example is a construction of a metabolism predictor. In specific examples, the metabolism predictor (e.g., metabolism model) uses machine learning algorithms and chemoinformatics to identify EC numbers and bacterial species (e.g., as shown in FIG. 2) related to the metabolism of a query molecule. In this particular example, metabolism predictor was used to identify microorganisms and enzymes belonging to gut microbiota. EC nomenclature identify classes of enzymes catalyzing similar reactions. The first number of the EC classification code represents the general type of reaction catalyzed by the enzyme and ranges from one to six ((Table 1). The following three numbers represent detailed reaction types. In this way, the second and third numbers are the enzyme's subclass and sub-subclass, respectively, and describe the reaction with regarding to the compound, group, bond or product involved in the reaction. The last number represents specific metabolites and cofactors involved in the reaction.

TABLE 1
Meaning of the first digit of EC nomenclature

| Reaction | | |
|---|---|---|
| Class | Name | Reaction catalyzed |
| 1 | Oxidoreductases | Redox (oxidation/reduction) reactions |
| 2 | Transferases | Transfer of a chemical group from one |
| | | molecule to another |
| 3 | Hydrolases | Hydrolysis: cleavage of a bond by |
| | | insertion of water |
| 4 | Lyases | Removal of a group with concomitant |
| | | formation of a double bond, or addition |
| | | of a group to a double bond |
| 5 | Isomerases | Isomerization of molecules (e.g., |
| | | racemases and epimerases) |
| 6 | Ligases | Joining of two molecules |

In specific examples, the method 100 can include one or more of: Step 1: Build an enzyme database. Obtain all proteome or proteins available from different sources. Then, from the proteins found, identify enzymes with an EC number associated. Finally, annotate those enzymes that do not have a proper EC associated using as base the identified enzymes.

In specific examples, the method 100 can include one or more of: Step 2: Build a substrate/product training dataset. From each knowing enzyme get the 3D structure of substrates and products involved in the reaction of all enzymes. Then, get structural features of substrate (e.g. product molecules, drugs) from different sources. Finally, perform a selection of important and relevant features for classification.

In specific examples, the method 100 can include one or more of: Step 3: Run a machine learning algorithm to classify and separate enzymes associated to the metabolism of the substrate (e.g. product molecules, drugs). Then using the substrate training dataset, optimize the parameters for machine learning algorithm. Next, construct and evaluate the machine learning classifier (build as many classifiers as needed, that is, 1, 2, . . . , n classifier). Finally, perform a prediction of the EC class and EC sub-class numbers for a query molecule.

In specific examples, the method 100 can include one or more of: Step 4: Obtain refined prediction of enzymes associated to the metabolism of a molecule, using structural similarity search and the known substrate dataset. Then, search for similar molecules for the query molecule using different coefficients of structural similarity. Next, filter according to different criteria of similarity. Finally, perform a reduction of the EC sub-sub-class and EC sub-sub-sub-class numbers for the query molecule.

In specific examples, the method 100 can include one or more of: Step 5: Assign an EC number, it means, a function, to each metabolic enzyme belonging to a species. Along with this, every gut bacteria involved in the metabolism of a query molecule will be also identified. From the EC number identified, obtain all metabolic enzyme and species. Finally, identify whose metabolic enzymes belonging to a gut bacteria species capable of metabolizing the query molecule. Assigning an EC number, means, a function, to each metabolic enzyme belonging to a specie. Along with this, every gut bacteria involved in the metabolism of a query molecule will be also identified.

An embodiment of a system for prediction of a protein function based on a protein feature in association with any multi-component protein-associated element (e.g. a query molecule).

The use of currently disclosed embodiment of a system, for metabolism of any query molecule, where a query molecule can include: drugs, other classes of xenobiotics (e.g. dietary compounds, environmental chemicals) and any other multi-component protein-associated, element.

In specific examples, metabolism predictor can be used for drug metabolism, but its use can be expanded to predict the metabolism of other classes of xenobiotics, such as dietary compounds, environmental chemicals, etc. As an embodiment of the technology, a set of gut bacterial species associated to Caffeine metabolism can be obtained with embodiments of the present technology method:

TABLE 2
Caffeine-degrading bacteria found using literature information and using
bioinformatics tools including machine learning and structural approaches
Bacteria found in literature:

Pseudomonas alcaligenes strain MTCC 5264
Pseudomonas putida
Pseudomonas fulva
Serratia marcescens
Pseudomonas putida No. 352
Pseudomonas sp. strain GSC 1182
Pseudomonas alcaligenes CFR 1708
Acetobacter sp. T3
Bacteria found with a metabolism model and/or
any suitable approaches described herein:
Streptococcus pneumoniae
Bacillus licheniformis
Bacillus megaterium
Bacillus subtilis
Pseudomonas putida
Pseudomonas aeruginosa
Brevibacillus laterosporus
Paenibacillus macerans
Aneurinibacillus migulanus
Cupriavidus metallidurans
Bacillus panaciterrae
Paenibacillus tianmuensis
Pseudomonas stutzeri
Paenibacillus assamensis
Paenibacillus lactis
Bacillus aquimaris
Bacillus endophyticus
Paenibacillus macquariensis
Paenibacillus polymyxa
Bacillus cereus
Bacillus thuringiensis
Bacillus tequilensis
Pseudomonas fluorescens
Alcanivorax sp. RHS-str. 303
Rhodoplanes sp. 303
Bacillus mojavensis
Bacillus flexus
Jeotgalibacillus marinus
Pseudomonas fulva

In specific examples, the drug metabolism predictor (e.g., metabolism model) was able to predict relationships between bacterial species and drugs already described in the literature. This is the case for Caffeine, where the species Pseudomonas putida and Pseudomonas fulva were predicted to be drug-degrading bacteria, as reported in the literature, along with a set of other bacteria non previously disclosed in relation with Caffeine.

Embodiment of the method and/or system, a set of bacterial species associated with inflammation can be obtained with embodiment of the present method and/or system:

TABLE 3
Butyrate-degrading bacteria were found using bioinformatics
tools including machine learning and structural approaches
Inflammation

| Bacteria found | |
|---|---|
| in literature: | produces: |
| [Eubacterium] rectale | butyrate |
| Coprococcus catus | butyrate |
| Roseburia intestinalis | butyrate |
| Anaerostipes | butyrate |
| Subdoligranulum variabile | butyrate |
| Roseburia hominis | butyrate |
| Roseburia faecis | butyrate |
| Roseburia inulinivorans | butyrate |
| Bacteroides uniformis | propionate |
| Bacteroides vulgatus | propionate |
| Veillonella | propionate |
| Coprococcus catus | propionate |
| Prevotella copri | propionate |
| Roseburia intestinalis | propionate |
| Dialister invisus | propionate |
| Akkermansia muciniphila | propionate |
| Roseburia inulinivorans | propionate |
| Dialister succinatiphilus | propionate |
| Phascolarctobacterium succinatutens | propionate |
| Faecalibacterium prausnitzii | polyamine |

Bacteria found with a metabolism model and/or
any suitable approaches described herein:

Streptococcus pneumoniae
Salmonella enterica
Citrobacter amalonaticus
Serratia fonticola
Enterobacter cloacae
Escherichia coli
Klebsiella oxytoca
Klebsiella pneumoniae
Marmoricola sp. S8-670
Vibrio parahaemolyticus
Citrobacter farmeri
Haemophilus influenzae

Embodiment of the method and/or system, a set of gut bacteria species associated with artificial sweeteners can be obtained with embodiments of the present method and/or system:

TABLE 4
Bacteria found in literature includes bacteria whose abundance levels are
increased or decreased due to the consumption of artificial sweeteners.
Saccharine-degrading bacteria were found using bioinformatics tools
including machine learning and structural approaches
Artificial Sweeteners

| Bacteria found | Consumption of | microbiota |
|---|---|---|
| in literature: | sweetener | effect |
| Enterobacteriaceae | Aspartame | increase |
| Anaerostipes | Saccharine | decrease |
| Ruminococcus | Saccharine | decrease |
| Adlercreutzia | Saccharine | decrease |
| Dorea | Saccharine | decrease |
| Deltaproteobacteria | Saccharine | increase |
| Enterobacteriaceae | Saccharine | increase |
| Sporosarcina | Saccharine | increase |
| Jeotgalicoccus | Saccharine | increase |
| Akkermansia | Saccharine | increase |
| Oscillospira | Saccharine | increase |
| Corynebacterium | Saccharine | increase |
| Roseburia | Saccharine | increase |
| Turicibacter | Saccharine | increase |
| Weissella | Saccharine | increase |
| Bacteroides vulgatus | Saccharine | increase |
| Bacteroides uniformis | Saccharine | increase |
| Bacteroides fragilis | Saccharine | increase |
| Bacteroides | Sucralose | decrease |
| Lactobacillus | Sucralose | decrease |
| Bifidobacterium | Sucralose | decrease |
| Streptococcus | Sucralose | decrease |
| Dehalobacterium | Sucralose | decrease |
| Anaerostipes | Sucralose | decrease |
| Ruminococcus | Sucralose | increase |
| Akkermansia | Sucralose | increase |
| Turicibacter | Sucralose | increase |
| Bacteroidetes: Firmicutes | Saccharine | decrease |

Bacteria found with a metabolism model and/or
any suitable approaches described herein:

Streptococcus pneumoniae
Bacillus cereus
Bacillus licheniformis
Bacillus megaterium
Bacillus subtilis
Bacillus thuringiensis
Bacillus tequilensis
Bacillus mojavensis
Bacillus flexus
Jeotgalibacillus marinus

In a specific example of the of the fourth module for personalized dietary recommendations, it shows an example of the advice that are given to individuals in terms of their vitamin levels. Embodiments of the method 100 and/or system 200 can include providing one or more recommendations associated with diet, food intake, and/or other associated aspects, such as based on one or more query molecule scores and/or other suitable data described herein.

Providing recommendations can include providing vitamin-related recommendations (e.g., notifications; information; etc.). In specific examples, providing vitamin-related recommendations can include providing one or more of verbal and/or graphical notifications including any suitable language including: “Vitamins are essential nutrients that your body needs to perform hundreds of important jobs, including building proteins and converting food into energy. Your cells can make some of these vitamins (such as vitamin D, if you get enough sun exposure), but most of these vitamins must come from other sources. Eating a well-balanced diet with lots of vitamin-rich foods—like fresh fruits and vegetables—provides the best supply for most of these vitamins. But did you know that your gut microbiome also produces certain vitamins? Let's explore your vitamin-producing bacteria, focusing on two important vitamins that your gut microbes can help supply: vitamin K and vitamin B9 (also called folate and folic acid). [Section Header] Your vitamin-producing bacteria: Abundance measures what portion of your microbiome a specific bacteria makes up. Below you can see the relative abundance of vitamin K-producing and vitamin B9-producing bacteria in your sample. [Graph title] ABUNDANCE. [Section title] VITAMIN K. Vitamin K is most widely known for its role in blood clotting, but it also plays other important roles in your body, such as helping to maintain strong bones and keeping your heart healthy. There are two types of vitamin K: vitamin K1 and K2. You can get vitamin K1 from green, leafy vegetables, vegetable oils, and some fruits. Vitamin K2, however, is mainly produced by bacteria in your gut. These vitamin K-producing bacteria use vitamin K1 to produce vitamin K2. Vitamin K2 is then absorbed into your body through the wall of your gut. [Graph title] YOUR VITAMIN K BACTERIA. [Sub-title] How you compare to all users. The abundance of your vitamin K-producing bacteria is greater than ______% {percentile} of selected users. [Sub-title] How you compare to selected samples. You have a {higher/lower} abundance of vitamin-K producing bacteria in your sample than our group of selected samples. Selected samples are samples from individuals who report no ailments and high levels of wellness. [Subheader]>Learn more If you are low (deficient) in vitamin K you may bruise more easily or experience nosebleeds and bleeding gums. Studies have also linked vitamin K deficiency to more serious health problems, like heart disease and osteoporosis. You can become deficient in Vitamin K if you don't get enough vitamin K from the food you eat or if you have a gut condition that limits the absorption of vitamin K. A shortage of certain gut bacteria may also play a role, as these bacteria help produce some of the vitamin K your body needs. See below for tips on how to increase the amount of vitamin K-producing bacteria in your gut. [Section Title] Vitamin B9 (folate, folic acid). Vitamin B9, also known as “folate” or “folic acid,” is involved in building and repairing DNA and forming new cells, such as red blood cells. While vitamin B9 is especially important during pregnancy, as it can help prevent birth defects in a baby's brain and spinal cord, it's also an essential nutrient throughout a person's life. There are many good dietary sources of vitamin B9. It is naturally present in several foods, including spinach, liver, garbanzo beans, asparagus, and brussels sprouts. It is also added to most grain-based products in the US, such as breads, cereals and pastas. Several gut bacteria also produce vitamin B9, providing an additional source of this important nutrient. [Graph title] YOUR VITAMIN B9 BACTERIA. [Sub-title] How you compare to all users The abundance of your vitamin K-producing bacteria is greater than ______% {percentile} of selected users. [Sub-title] How you compare to selected samples: You have a {higher/lower} abundance of vitamin-B9 producing bacteria in your sample than our group of selected samples. Selected samples are samples from individuals who report no ailments and high levels of wellness. [Subheader]>Learn more: If you have too little vitamin B9, you can develop a condition called megaloblastic anemia. Symptoms of megaloblastic anemia include fatigue, weakness, difficulty concentrating, headaches, irritability, heart palpitations, and shortness of breath. A vitamin B9 deficiency can also cause other problems, such as a sore tongue or mouth. Studies have shown that higher levels of this nutrient may be linked with improved quality of sleep. Research also suggests that vitamin B9 might help protect against depression and mental decline as we age. [Section title] TAKE ACTION: Below are some suggestions of ways to take action and increase the abundance of specific microbes. You don't need to take all these steps—simply pick the suggestions that work best for you and your lifestyle. All of these suggestions are based on scientific research. In case you want to learn more about these studies, we list the published papers at the bottom of the page. [Recommendations to be inserted, based on each user's results] IF: Vitamin K metabolism is LOW AND Lactococcus lactis is low: Consuming certain dairy products—such as buttermilk, sour cream, cottage cheese, and kefir—can boost your supply of a vitamin K-producing bacterium called Lactococcus lactis (subspecies lactis or cremoris). Be sure to check the label of these products to make sure they contain live cultures of this bacterium. AND Bacillus is low Japanese natto is an excellent source of a vitamin K-producing bacterium called Bacillus subtilis. This is a traditional Japanese food made from fermented soybeans. Research suggests that Japanese women who regularly consumed natto had higher vitamin K levels than women who did not eat natto very often. Taking probiotic supplements is another way to boost your vitamin K-producing bacteria. Research suggests that taking a supplement that contains Bacillus subtilis every day can increase levels of this helpful microbe. Check the label to make sure the supplement contains at least 10×10\hat{ }9 CFU of this bacterium. AND Prevotella is low: Adopting a Mediterranean diet may help boost your vitamin K-producing bacteria. Research suggests that people who follow a Mediterranean style diet have higher levels of a vitamin K-producing bacteria called Prevotella. This type of diet primarily consists of fresh fruits and vegetables, plant-derived oils (such as olive oil), seeds, nuts, fish, and legumes. It is low in saturated fats (such as butter), dairy, and red meat. IF: Vitamin B9 Metabolism is LOW AND Bacteroides intestinalis is low Xylan is a type of complex sugar (a polysaccharide) found in the cell walls of plants. Research shows that it can help increase your levels of a B9-producing bacteria called Bacteroides intestinalis. Xylan is most abundant in grains such as wheat, oats, rice, corn, barley, rye, and millet. Dietary guidelines recommend eating at least 6 ounces of grains per day. AND Rumino coccus is low: Research suggests that eating more dietary fiber can increase the amount of a vitamin B9-producing bacteria called Ruminococcus. Good sources of fiber include beans, whole grains, brown rice, nuts, and vegetables. Eating foods made with rice bran may also boost your supply of Ruminococcus. One study found that consuming 30 grams of rice bran per day increased levels of these vitamin-producing microbes. AND Anaerostipes is low: Inulin is a type of plant fiber found in many foods, including bananas, asparagus, onions, and artichokes. It is also available as a prebiotic supplement. Research suggests that taking an inulin supplement daily for at least four weeks can increase a vitamin B9-producing bacteria called Anaerostipes. The recommended dose for inulin is up to 10 grams per day. AND Blautia hydrogenotrophica is low: Xylooligosaccharide (XOS) is another prebiotic supplement that can boost vitamin B9-producing bacteria. One study found that consuming 2 grams of XOS per day for at least eight weeks can increase one B9-producing bacteria called Blautia hydrogenotrophica. AND Bifidobacterium is low: There are several things you can do to increase your levels of Bifidobacterium, another bacterial genus associated to production of vitamin B9: Consume inulin (recommended intake: 12-20 g/day) for at least 4 weeks. You can obtain inulin from commercially available prebiotic products or certain foods, such as globe artichoke, asparagus, bananas, bitter gourd, chicory root, endive, jerusalem artichoke, lettuce, onion, peach, peas, pomegranate, root vegetables, watermelon, shallot, whole grain wheat, whole grain rye, and soft-necked garlic. Consume dietary fiber (recommended intake: 17-30 g/day) for at least 28 days. The main sources of dietary fiber are whole-grain cereals, fruit, vegetables, and legumes. Consume a mixture of inulin and oligofructose at a 1:1 ratio (recommended intake: 6-16 g/day) for at least ₃ weeks. Consume a variety of fiber-rich foods to increase your Bifidobacterium levels. Try consuming whole-grain breakfast cereals (recommended intake: 48 g/day) for at least 3 weeks. Consume galacto-oligosaccharides (GOS) (recommended intake: 8-15 g/day) for at least 21-36 days. You can obtain GOS from commercially available prebiotic supplements or by consuming foods containing GOS, such as a variety of legumes and some milk powders. Consume wheat bran extract (10 g/day) for at least 3 weeks. Consume arabinoxylan oligosaccharides (AXOS) (recommended intake: 4.8 g/day) for at least 3 weeks. AXOS can be found in many products containing whole grain wheat. Consume xylooligosaccharides (XOS) (recommended intake: 1.2-2.8 g/day) for at least 3 weeks. You can obtain XOS from commercially available prebiotic products. Consume prebiotic fructans, which can be found in agave, (recommended intake: 5 g/day) for at least 3 weeks. The recommended healthy intake of fruit is 2 cups per day. Try including apples and kiwifruit in your diet!”

TABLE 5
Microorganism taxa associated with vitamins.

| Bacteria | Association |
|---|---|
| Anaerostipes caccae | Vitamin B9 |
| Bacteroides cellulosilyticus | Vitamin B9 |
| Bacteroides intestinalis | Vitamin B9 |
| Bacteroides vulgatus | Vitamin B9 |
| Bifidobacterium | Vitamin B9 |
| Bifidobacterium animalis | Vitamin B9 |
| Bifidobacterium asteroides | Vitamin B9 |
| Bifidobacterium longum | Vitamin B9 |
| Blautia hydrogenotrophica | Vitamin B9 |
| Parabacteroides distasonis | Vitamin B9 |
| Parabacteroides johnsonii | Vitamin B9 |
| Parabacteroides merdae | Vitamin B9 |
| Ruminococcus sp. | Vitamin B9 |
| Bacillus | vitamin K |
| Bacillus coagulans | vitamin K |
| Bacillus sp. LCP35 | vitamin K |
| Bacillus sp. P109 | vitamin K |
| Bacillus sp. SG23 | vitamin K |
| Bacillus sp. SGE126(2010) | vitamin K |
| Desulfovibrio | vitamin K |
| Desulfovibrio desulfuricans | vitamin K |
| Desulfovibrio sp. feline | |
| oral taxon 347 | vitamin K |
| Desulfovibrio sp. UNSW3caefatS | vitamin K |
| Enterococcus | vitamin K |
| Enterococcus faecalis | vitamin K |
| Enterococcus faecium | vitamin K |
| Enterococcus hermanniensis | vitamin K |
| Lactococcus lactis | vitamin K |
| Prevotella | vitamin K |
| Prevotella genomosp. P4 | vitamin K |
| Prevotella melaninogenica | vitamin K |
| Prevotella nigrescens | vitamin K |
| Prevotella oris | vitamin K |
| Prevotella salivae | vitamin K |
| Prevotella sp. canine oral taxon 195 | vitamin K |
| Prevotella sp. P2A_FAAD4 | vitamin K |
| Veillonella | vitamin K |
| Veillonella rodentium | vitamin K |

In a specific example of the of the fourth module for personalized dietary recommendations, it shows an example of the advice that are given to individuals in terms of their metabolism.

Providing recommendations can include providing metabolism-related recommendations (e.g., notifications, information, etc.). In specific examples, providing metabolism-related recommendations can include providing one or more of verbal and/or graphical notifications including any suitable language including: “You've probably heard people talk about their metabolism in relation to how quickly they burn calories—“I have a slow (or fast) metabolism.” Your metabolism is much more than this! It includes all the biochemical processes involved in converting what you take in into energy and producing the compounds your cells need to survive. It's a big job, and your microbiome plays a key role. Microbes in your gut specialize in digesting molecules that your body is unable to digest on its own. After breaking down these molecules into smaller chunks, the microbes use these smaller pieces to build unique molecules that your body cannot make by itself. Some of these molecules serve as fuel for your cells, while others perform more specialized roles, such as providing chemical signals that help regulate your gut health, appetite, and immune system. Let's explore how your microbiome supports metabolism by looking at the microbes that help you metabolize three types of molecules: carbohydrates, lipids, and amino acids. [Graph title] ABUNDANCE: Abundance measures what portion of your microbiome a specific bacteria makes up. Below, you can see the relative abundance of carbohydrate-, amino acid-, and lipid-metabolizing bacteria in your sample. Carbohydrates and your microbes: Carbohydrates are the main source of energy for your cells. They include sugars, starches, and fibers and are typically found in fruits, grains, vegetables, and milk products. Your cells are able to directly break down simple carbohydrates on their own, such as the sugars in fruit (fructose) and candy (glucose). Help is required, however, with the complex carbohydrates from plant-based foods, like fibers and starches. That's where your gut microbes pitch in, helping to break down these complex carbohydrates and convert them into energy and useful molecules. One of the molecules they produce is butyrate. This is a type of helpful fat known as a short chain fatty acid (SCFA). Studies have linked higher levels of butyrate with a lower risk of Crohn's disease. Higher levels of this fatty acid are also associated with a decrease in appetite-boosting hormones, as well as a lower risk of obesity. This suggests that feeding your carbohydrate-consuming bacteria with certain complex carbohydrates they love, either through food or supplements, may help you feel fuller after a meal and help you manage your weight. Microbes that help break down complex carbohydrates include bacteria such as Bacteroides, Roseburia, Butyrivibrio, Ruminococcus, Bifidobacterium, and Prevotella. Below, we provide suggestions on how you can boost your abundance of these helpful carbohydrate consumers. [Graph title] YOUR CARBOHYDRATE-METABOLIZING BACTERIA [Sub-title] How you compare to all users: The abundance of your carbohydrate-metabolizing bacteria is greater than ______% {percentile} of all users. [Sub-title] How you compare to selected samples: You have a {higher/lower} abundance of carbohydrate-metabolizing bacteria in your sample than our group of selected samples. Selected samples are samples from individuals who report no ailments and high levels of wellness. Lipids and your microbes: Another word for lipids is “fats,” and fats are an essential part of a healthy diet. For example, you need fats to build cell membranes, store energy, and help create hormones—including a hormone that helps to control appetite. Your lipid-consuming microbes help your body use fats in a few important ways. First, they help your body break down fats. They also use smaller molecules to make fats called short chain fatty acids (SCFA), including an important one called butyrate (see above). SCFAs provide fuel for the cells lining your gut and serve as messenger molecules that can communicate with other organs. High levels of SCFAs have been linked to a healthy gut and immune system. Finally, your gut microbes play a role in the levels of lipids that end up in your blood. For example, a group of microbes called Christensenella is associated with lower levels of triglycerides. If your triglycerides are continually high, this can increase your risk of having a stroke. However, your lipid-consuming microbes aren't always so helpful. Another group, called Eggerthella, is associated with an increase in triglyceride levels and a decrease in levels of “good cholesterol” called high-density lipoproteins (HDL), which help protect against heart disease. Below, we provide tips on how you can boost your abundance of helpful lipid-consuming microbes. [Graph title] YOUR LIPID-METABOLIZING BACTERIA: [Sub-title] How you compare to all users: The abundance of your lipid-metabolizing bacteria is greater than ______% {percentile} of selected users. [Sub-title] How you compare to selected samples. You have a {higher/lower} abundance of lipid-metabolizing bacteria in your sample than our group of selected samples. Selected samples are samples from individuals who report no ailments and high levels of wellness. Amino acids and your microbes: Amino acids are the building blocks of proteins, which play a critical role in producing muscle, bone, cartilage, skin and blood in the body. There are 21 amino acids in the body that form the proteins necessary for human life and health. Many of these amino acids can be created in your body from other amino acids. But nine of them cannot be created in this way—we call these “essential” amino acids because they are necessary for human life, but we can't produce them in our bodies. Instead, we rely on foods (and sometimes dietary supplements) to obtain these, with help from our gut microbes. During the digestive process, gut microbes go to work breaking down some of the proteins from your food into essential amino acids for your body. One key amino acid your gut microbes produce is tryptophan. Your cells use tryptophan to create serotonin, an important chemical messenger for your brain and nerves. Serotonin can influence your social behavior and is often associated with emotional wellness. Research suggests that as much as 90 percent of serotonin is produced in the gut, and much of that is regulated by your gut microbes. Below, we provide tips on how you can boost your abundance of helpful amino acid microbes. [Graph title] YOUR AMINO ACID-METABOLIZING BACTERIA [Sub-title] How you compare to all users. The abundance of your amino acid-metabolizing bacteria is greater than ______% {percentile} of selected users. [Sub-title] How you compare to selected samples. You have a {higher/lower} abundance of amino acid-metabolizing bacteria in your sample than our group of selected samples. Selected samples are samples from individuals who report no ailments and high levels of wellness. Recommendations/Take Action: Below are some suggestions of ways to take action and increase the abundance of specific metabolism microbes. You don't need to take all these steps—simply pick the suggestions that work best for you and your lifestyle. All of these suggestions are based on scientific research. In case you want to learn more about these studies, we list the published papers at the bottom of the page. [Recommendations to be inserted from spreadsheet, based on each user's results]. IF: CARBOHYDRATE METABOLISM IS LOW AND Anaerostipes is low: Consume inulin (recommended intake: 12 g/day) for at least 4 weeks to increase Anaerostipes. You can obtain inulin from commercially available prebiotic products, there are also foods that contain it, such as globe artichoke, asparagus, bananas, bitter gourd, chicory root, endive, jerusalem artichoke, lettuce, onion, peach, peas, pomegranate, root vegetables, watermelon, shallot, whole grain wheat, whole grain rye, soft-necked garlic. IF: CARBOHYDRATE metabolism is LOW AND/OR LIPID metabolism is LOW: AND Coprococcus is low: Consume a mixture of inulin and oligofructose at a 1:1 ratio (recommended intake: 6-16 g/day) for at least 3 weeks to increase Coprococcus. This microorganism is involved in metabolizing carbohydrates and lipids and improves your gut microbiota's carbohydrate and lipid metabolism. AND Dorea is low: Consume a mixture of inulin and oligofructose at a 1:1 ratio (recommended intake: 6-16 g/day) for at least 3 weeks to increase Dorea. This microorganism is involved in metabolizing carbohydrates and lipids and improves your gut microbiota's carbohydrate and lipid metabolism. AND Lactobacillus is low: To increase the amount of Lactobacillus in your sample you can: Consume inulin (recommended intake: 10 g/day) for at least 3 weeks . You can obtain inulin from commercially available prebiotic products, there are also foods that contain it, such as globe artichoke, asparagus, bananas, bitter gourd, chicory root, endive, jerusalem artichoke, lettuce, onion, peach, peas, pomegranate, root vegetables, watermelon, shallot, whole grain wheat, whole grain rye, soft-necked garlic. Consume different fiber-rich foods. You can consume whole grain oat-based granola (recommended intake: 45 g/day) for at least 4 weeks or whole-grain breakfast cereals (recommended intake: 48 g/day) for at least 3 weeks. Consume galacto-oligosaccharides (GOS) (recommended intake: up to 15 g/day) for at least 36 days. You can obtain GOS from commercially available prebiotic supplements or by consuming foods containing GOS, such as a variety of legumes and some milk powders. Consume xylooligosaccharides (XOS) (recommended intake: approx. 1.2 g/day) for at least 3 weeks. You can obtain XOS from commercially available prebiotic products. Consume prebiotic fructans, which can be found in agave, (recommended intake: 5 g/day) for at least 3 weeks. The recommended healthy intake of fruit is 2 cups per day. Try including apples and kiwifruit in your diet! These microorganisms are involved in metabolizing carbohydrates and lipids and improve your gut microbiota's carbohydrate and lipid metabolism. AND Oscillospira is low Consume a mixture of inulin and oligofructose at a 1:1 ratio (recommended intake: 6-16 g/day) for at least 3 weeks to increase Oscillospira. This microorganism is involved in metabolizing carbohydrates and lipids and improves your gut microbiota's carbohydrate and lipid metabolism. IF: LIPID metabolism is LOW AND/OR AMINO ACID metabolism is LOW AND Bacteroides is low: Consume xylooligosaccharides (XOS) (recommended intake: 2.8 g/day) for at least 8 weeks to increase the levels of some species of Bacteroides. You can obtain XOS from commercially available prebiotic products. These microorganisms are involved in metabolizing amino acids and lipids and improve your gut microbiota's amino acid and lipid metabolism. IF: CARBOHYDRATE metabolism is LOW AND/OR LIPID metabolism is LOW AND/OR AMINO ACID metabolism is LOW AND Bifidobacterium is low: There are several things you can do to increase your levels of Bifidobacterium: Consume inulin (recommended intake: 12-20 g/day) for at least 4 weeks. You can obtain inulin from commercially available prebiotic products or certain foods, such as globe artichoke, asparagus, bananas, bitter gourd, chicory root, endive, jerusalem artichoke, lettuce, onion, peach, peas, pomegranate, root vegetables, watermelon, shallot, whole grain wheat, whole grain rye, and soft-necked garlic. Consume dietary fiber (recommended intake: 17-30 g/day) for at least 28 days. The main sources of dietary fiber are whole-grain cereals, fruit, vegetables, and legumes. Consume a mixture of inulin and oligofructose at a 1:1 ratio (recommended intake: 6-16 g/day) for at least ₃ weeks. Consume a variety of fiber-rich foods to increase your Bifidobacterium levels. Try consuming whole-grain breakfast cereals (recommended intake: 48 g/day) for at least 3 weeks. Consume galacto-oligosaccharides (GOS) (recommended intake: 8-15 g/day) for at least 21-36 days. You can obtain GOS from commercially available prebiotic supplements or by consuming foods containing GOS, such as a variety of legumes and some milk powders. Consume wheat bran extract (recommended intake: 10 g/day) for at least 3 weeks. Consume arabinoxylan oligosaccharides (AXOS) (recommended intake: 4.8 g/day) for at least 3 weeks. AXOS can be found in many products containing whole grain wheat. Consume xylooligosaccharides (XOS) (recommended intake: 1.2-2.8 g/day) for at least 3 weeks. You can obtain XOS from commercially available prebiotic products. Consume prebiotic fructans, which can be found in agave, (recommended intake: 5 g/day) for at least 3 weeks. The recommended healthy intake of fruit is 2 cups per day. Try including apples and kiwifruit in your diet! These microorganisms are involved in metabolizing amino acids, carbohydrates, and lipids and can help improve your gut microbiota's metabolism!”

TABLE 6
Microorganism taxa associated with metabolism-related aspects

| Bacteria | Association |
|---|---|
| Anaerostipes | Carbohydrate Metabolism |
| Bifidobacterium | Carbohydrate Metabolism |
| Blautia | Carbohydrate Metabolism |
| Coprococcus | Carbohydrate Metabolism |
| Dialister | Carbohydrate Metabolism |
| Dorea | Carbohydrate Metabolism |
| Eubacterium | Carbohydrate Metabolism |
| Faecalibacterium | Carbohydrate Metabolism |
| Lactobacillus | Carbohydrate Metabolism |
| Lactococcus | Carbohydrate Metabolism |
| Odoribacter | Carbohydrate Metabolism |
| Oscillospira | Carbohydrate Metabolism |
| Phascolarctobacterium | Carbohydrate Metabolism |
| Veillonella | Carbohydrate Metabolism |
| Bacteroides | Lipid Metabolism |
| Bifidobacterium | Lipid Metabolism |
| Bilophila | Lipid Metabolism |
| Blautia | Lipid Metabolism |
| Butyricimonas | Lipid Metabolism |
| Coprococcus | Lipid Metabolism |
| Dorea | Lipid Metabolism |
| Eggerthella | Lipid Metabolism |
| Holdemania | Lipid Metabolism |
| Lachnospira | Lipid Metabolism |
| Lactobacillus | Lipid Metabolism |
| Odoribacter | Lipid Metabolism |
| Oscillospira | Lipid Metabolism |
| Ruminococcus | Lipid Metabolism |
| Bacillus | Amino acid Metabolism |
| Bacteroides | Amino acid Metabolism |
| Bifidobacterium | Amino acid Metabolism |
| Escherichia | Amino acid Metabolism |
| Hafnia | Amino acid Metabolism |
| Klebsiella | Amino acid Metabolism |
| Lactococcus | Amino acid Metabolism |
| Morganella | Amino acid Metabolism |
| Odoribacter | Amino acid Metabolism |
| Propionibacterium | Amino acid Metabolism |
| Staphylococcus | Amino acid Metabolism |
| Streptococcus | Amino acid Metabolism |

In a specific example of the of the fourth module for personalized dietary recommendations, it shows an example of the advices that are given to individuals in terms of artificial sweeteners levels intake.

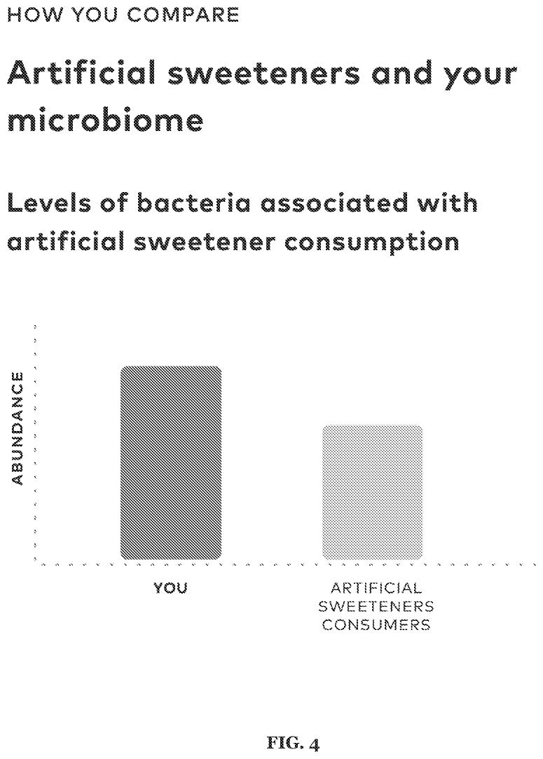

Providing recommendations can include providing artificial sweetener-related recommendations (e.g., notifications, information, specific example as shown in FIG. 4, etc.). In specific examples, providing artificial sweetener-related recommendations can include providing one or more of verbal and/or graphical notifications including any suitable language including: Artificial Sweeteners Explorer: Introduction: “Artificial sweeteners may not be so sweet after all. Research suggests that, while these sugar substitutes cut calories, there may be a cost to both your gut microbiome and overall wellness. In this section, we look at the levels of your bacteria that may be affected by the artificial sweeteners aspartame, saccharin, and sucralose and explore ways to potentially maintain (or regain) your microbial balance. What are artificial sweeteners? Artificial sweeteners, such as aspartame, saccharin, and sucralose, are sugar substitutes that provide a sugar-like sweetness with few or no calories. They are among the most commonly used food additives, appearing on the labels of a wide variety of foods and drinks, including reduced-calorie sodas, sports drinks, yogurts, cereals, and desserts, as well as many other “diet,” “sugar free,” and “no sugar added” products. They are also found in several everyday items you might not expect, such as toothpaste, mouthwash, and some vitamins and medications. Foods and drinks with artificial sweeteners may seem like an obvious choice if you are trying to cut down on calories and sugar. However, studies suggest these sugar substitutes may actually increase the chance of weight gain, as well as Type 2 diabetes and other metabolic problems. Scientists think the reasons behind this are complex, relating to how the body and brain—as well as the microbiome—respond to these sweeteners. DID YOU KNOW? Artificial sweeteners are so widely used in foods and drinks that you may be ingesting these sugar substitutes even if you never intentionally consume a “diet” or “low calorie” product. In one small study, for example, artificial sweeteners were found in the breast milk of women who did not recall consuming any foods or drinks with these sweeteners. In another study, 8 of 18 people who reported never consuming artificial sweeteners still had detectable sucralose in their urine. Not so sweet for your microbiome? Most artificial sweeteners pass through your gastrointestinal tract without being broken down or absorbed—and instead directly encounter your gut microbes. Studies suggest these sweeteners may alter the balance of bacterial species in your gut, with potential effects on your wellness. In a human study, long-term use of artificial sweeteners was associated with greater populations of bacteria from the Enterobacteriaceae family, the Deltaproteobacteria class, and the Actinobacteria phylum. Use of artificial sweeteners was also associated with increased weight and blood glucose levels. So far, however, most of the research on these questions has been in lab animals. In animal studies, artificial sweeteners seem to affect the balance of two large groups of gut bacteria—Firmicutes and Bacteroidetes—that have been linked to weight gain and loss. Preliminary research suggests artificial sweeteners may promote the growth of Firmicutes at the expense of Bacteroidetes. Microbiomes tilted in favor of Firmicutes tend to be associated with weight gain. Based on the animal and human studies described above, scientists think artificial sweeteners may prompt an imbalance (called dysbiosis) in the gut microbiome. The consequences of this dysbiosis are still being explored. Research in mice and humans also suggests that artificial sweeteners may alter the microbiome in ways that increase the risk of glucose intolerance (higher-than-normal blood sugar levels). This metabolic condition can be a precursor to Type 2 diabetes and is a risk factor for heart disease. Artificial sweeteners and your microbiome: Use of artificial sweeteners has been associated with greater levels of several kinds of bacteria. We've compared the total abundance of these bacteria in your sample with the abundance in people who report using artificial sweeteners. Surprised by your results? If your levels of these bacteria are higher than you anticipated, keep in mind that artificial sweeteners are in many products you might not expect, including some cereals and sports drinks. You may be regularly consuming these sweeteners without knowing it. Next time you go to the grocery store, consider taking a close look at the labels on foods and drinks to see where these sweeteners are hiding. You can take steps to decrease the abundance of these bacteria. We provide tips in the Take Action section below. More about these sweeteners: Sucralose (Splenda), saccharin (Sweet'N Low), and aspartame (NutraSweet, Equal) are among the most widely used artificial sweeteners, with surveys suggesting that one-third of US adults regularly consume foods and drinks with these sugar substitutes. Some familiar products that contain these sweeteners include Diet Coke (aspartame), Diet Mountain Dew (saccharin and aspartame), Fiber One Original Bran Cereal (sucralose), Gatorade G2 (sucralose), Yoplait Light Yogurt (sucralose), and many Crest and Colgate toothpastes (saccharin). TAKE ACTION: Your microbiome is dynamic and can respond quickly to changes in what you eat and drink. If you think artificial sweeteners could be affecting your levels of certain bacteria, you might try eliminating these sweeteners from your diet for a few weeks. You can then send in another Explorer sample to see if your levels have changed. Just make sure you closely check all food and drink labels—artificial sweeteners may be hiding in products you don't expect, such as cereal, yogurt, and sports drinks. We've also provided some ways to decrease your levels of certain bacteria that may have been increased by artificial sweeteners. These recommendations are based on which types of bacteria were increased in your sample, compared with people who report using artificial sweeteners. You don't need to take all these steps—simply pick the suggestions that work best for you and your lifestyle. You might try one of these steps at a time to see what works for you. To decrease your levels of Enterobacteriaceae, you can: Consume a variety of fiber-rich foods. Good sources of fiber include beans, brown rice, nuts, vegetables, and whole grains. You might try eating a whole-grain breakfast cereal for at least ₃ weeks (recommended intake: 1¾ cups per day). Consume more food containing inulin. You can get inulin (a soluble plant fiber) from many foods, including globe artichokes, asparagus, bananas, chicory root, endive, Jerusalem artichokes, lettuce, onions, peaches, peas, pomegranates, root vegetables, watermelon, shallots, whole grain wheat, whole grain rye, and garlic. You can also obtain inulin from prebiotic products, such as powders and supplements. Consume more food with soluble and insoluble dietary fiber. Good sources of soluble fiber include apples, citrus fruits, beans, peas, carrots, oats, and barley. Good sources of insoluble fiber include whole-wheat flour, nuts, and vegetables, such as beans, cauliflower, and potatoes. To decrease your levels of Deltaproteobacteria, you can: Avoid the consumption of a Western diet, especially foods high in milk-derived saturated fats, such as butter.

Example Taxa associated with one or more variants (e.g., where any combination of one or more taxa can be associated with, such as positively correlated with, negatively correlated with, and/or otherwise associated with one or more artificial sweetener-related conditions and/or variants of the technology) can include Enterobacteriaceae (family); Deltaproteobacteria (class); and/or Actinobacteria (phylum).

Any suitable recommendations herein can include one or more therapeutic recommendations (e.g., probiotic compositions, prebiotic compositions, microbiome-modifying therapeutic recommendations, such as based on a query molecule score, etc.)

In a specific example of the of the fourth module for personalised dietary recommendations, it shows an example of the advices that are given to individuals in terms of alcohol levels intake.

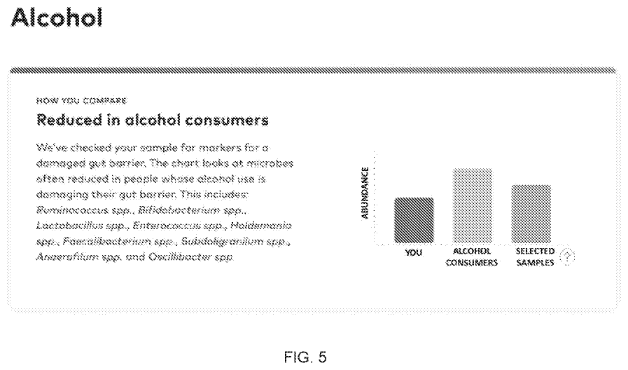

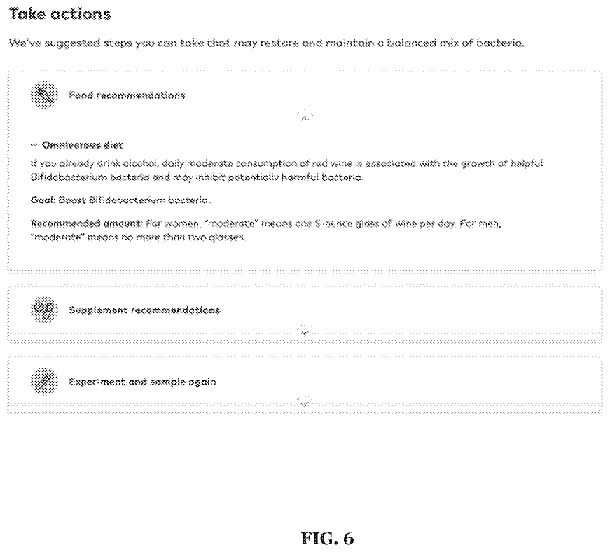

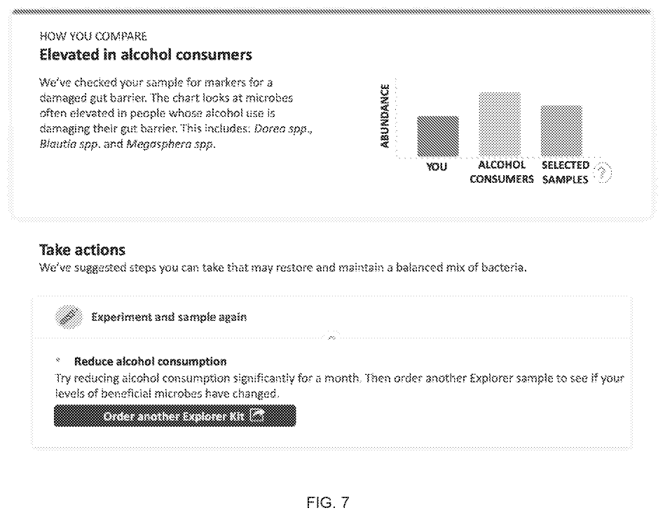

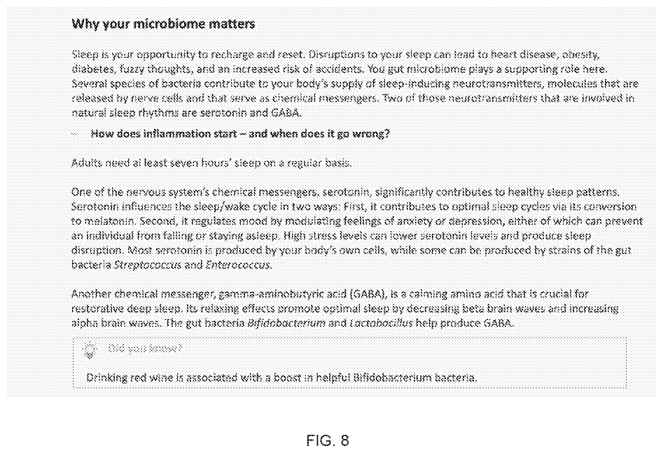

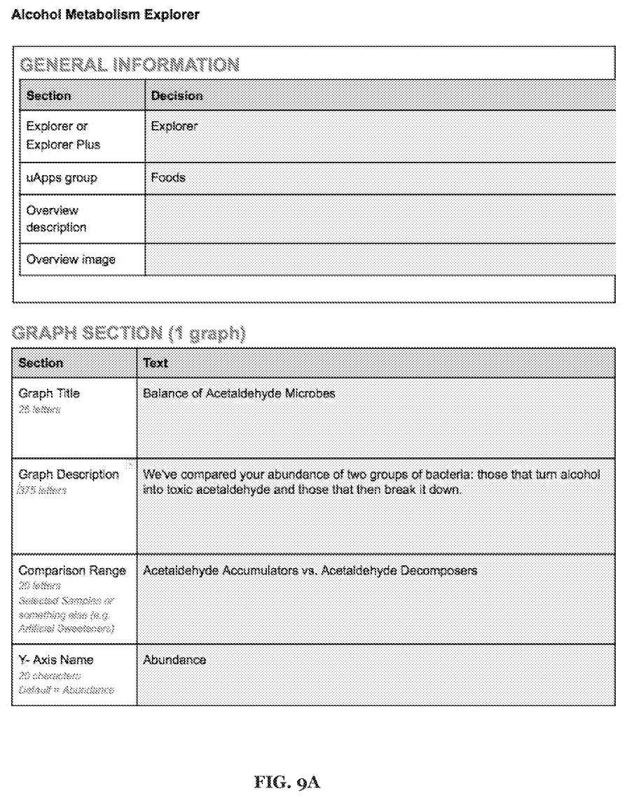

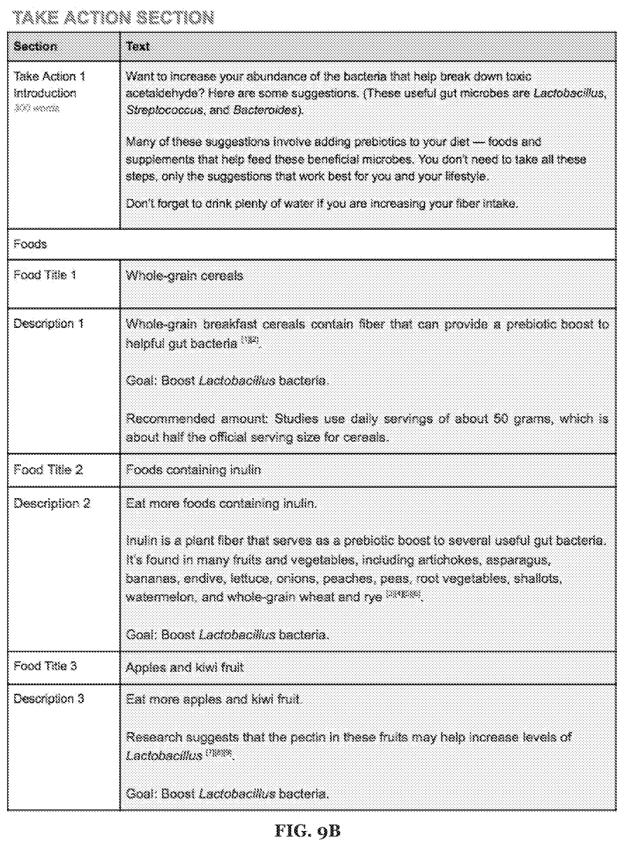

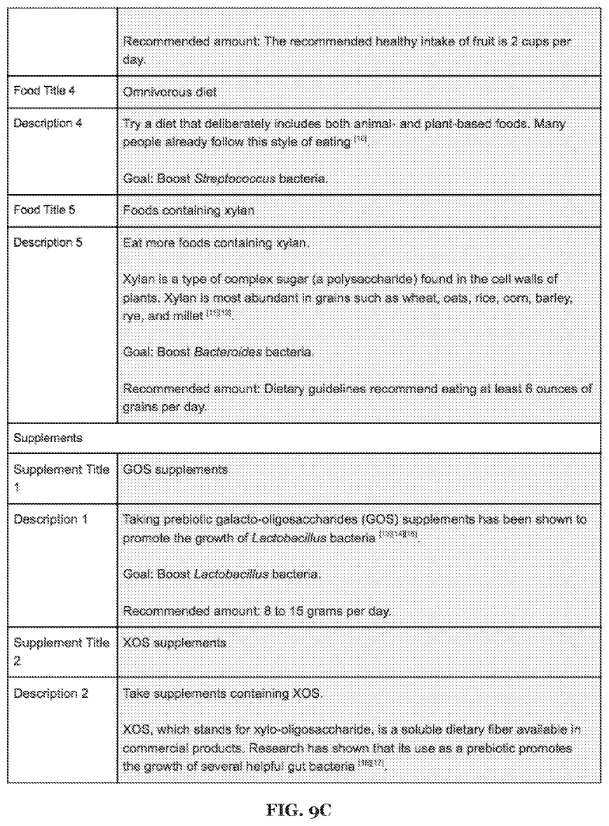

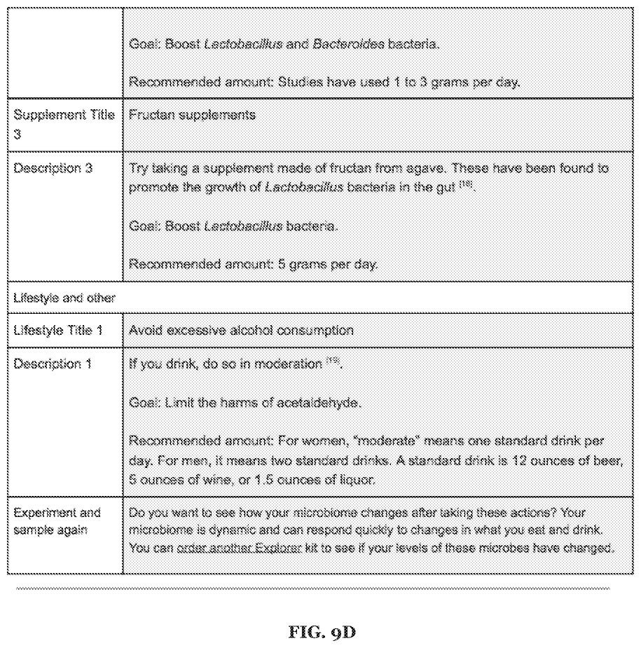

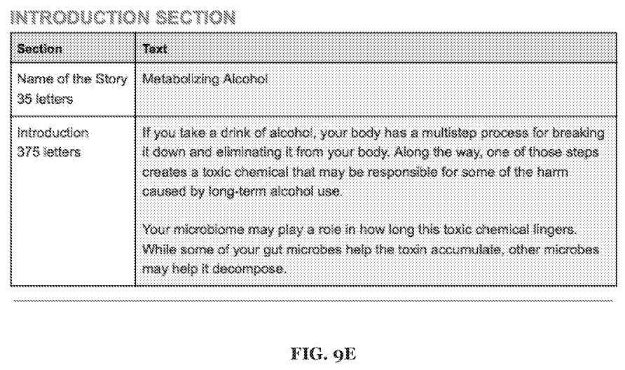

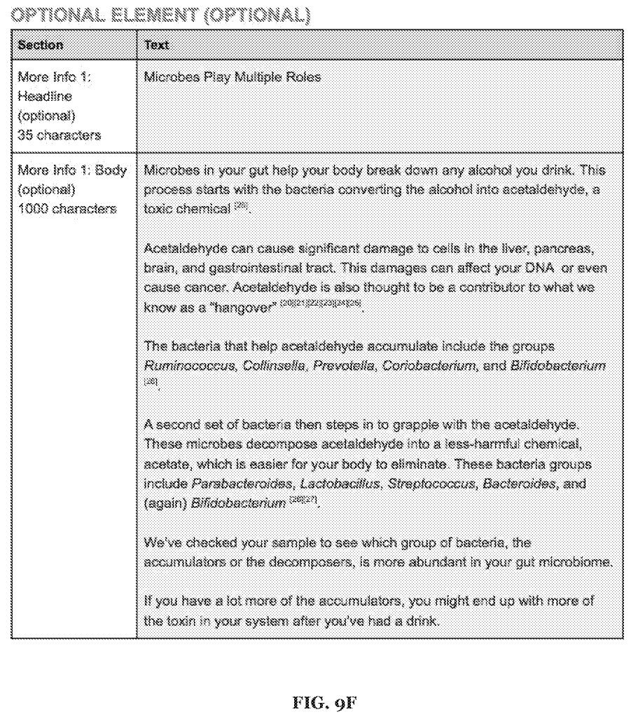

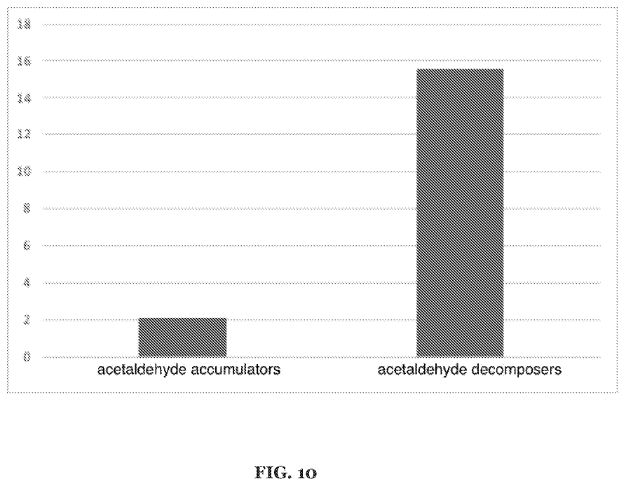

Providing recommendations can include providing alcohol-related recommendations (e.g., alcohol-related and/or alcohol metabolism-related data, information, and/or recommendations; specific examples as shown in FIGS. 5-8, 9A-9F, and 10, etc.). In specific examples, providing alcohol-related recommendations can include providing one or more of verbal and/or graphical notifications including any suitable language. Example Taxa associated with one or more of alcohol metabolism and/or one or more variants (e.g., where any combination of one or more taxa can be associated with, such as positively correlated with, negatively correlated with, and/or otherwise associated with one or more artificial sweetener-related conditions and/or variants of the technology) can include Bacteroides uniformis (species); Holdemania filiformis (species); Turicibacter sanguinis (species); Eisenbergiella tayi (species); Erysipelatoclostridium ramosum (species); Dielma fastidiosa (species); Roseburia hominis (species); Catenibacterium mitsuokai (species); Solobacterium moorei (species); Eggerthia catenaformis (species); Allobaculum stercoricanis (species); Lactobacillus (genus);

Embodiments of the system and/or platform can include every combination and permutation of the various system components and the various platform processes, including any variants (e.g., embodiments, variations, examples, specific examples, figures, etc.), where portions of embodiments of the method and/or processes described herein can be performed asynchronously (e.g., sequentially), concurrently (e.g., in parallel), or in any other suitable order by and/or using one or more instances, elements, components of, and/or other aspects of the system and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the platform and/or system can be embodied and/or implemented at least in part as a machine configured to receive a computer-readable medium storing computer-readable instructions. The instructions can be executed by computer-executable components that can be integrated with embodiments of the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the system, platform, and/or variants without departing from the scope defined in the claims. Variants described herein not meant to be restrictive. Certain features included in the drawings may be exaggerated in size, and other features may be omitted for clarity and should not be restrictive. The figures are not necessarily to scale. Section titles herein are used for organizational convenience and are not meant to be restrictive. The description of any variant is not necessarily limited to any section of this specification.

We claim:

1. A method for metabolism-related prediction, the method comprising:
generating an enzyme dataset comprising:
enzyme data indicating a set of enzymes associated with a set of microorganism taxa, and
chemical reaction data associated with the set of enzymes;
generating a substrate dataset comprising substrate structural features associated with a set of substrates actable upon by the set of enzymes;
generating a machine learning model for predicting an enzyme feature associated with a metabolism of a query molecule, based on the enzyme dataset and the substrate dataset;
determining a microorganism taxon associated with the metabolism of the query molecule based on the enzyme feature predicted from the machine learning model; and
determining a query molecule score for a user based on the microorganism taxon and a microbiome characterization for the user, wherein the query molecule score is associated with the query molecule.

2. The method of claim 1, wherein the query molecule comprises a drug, and wherein the query molecule score comprises a drug score indicating a drug efficacy for the user for the drug.

3. The method of claim 2, further comprising promoting a therapy to the user for a microorganism-related condition based on the drug score.

4. The method of claim 3, wherein promoting the therapy comprises providing a recommendation for the therapy to the user.

5. The method of claim 1, wherein the substrate structural features comprise at least one of 3D structural features associated with the set of substrates, product molecule features associated with the set of substrates, and drug features associated with the set of substrates.

6. The method of claim 5, further comprising, for each substrate of the set of substrates, identifying a subset of relevant features from the 3D structural features, the product molecule features, and the drug features, wherein generating the machine learning model comprises generating the machine learning model for predicting the enzyme feature associated with metabolism of the query molecule based on the enzyme dataset and the subset of relevant features.

7. The method of claim 1, wherein the chemical reaction data comprises Enzyme Commission number data associated with the set of enzymes, and wherein the enzyme feature comprises an Enzyme Commission number feature for the query molecule.

8. The method of claim 7, wherein the set of enzymes comprises a first subset of enzymes unassociated with the Enzyme Commission number data and a second subset of enzymes associated with the Enzyme Commission number data, and wherein generating the enzyme dataset comprises annotating the first subset of enzymes based on the Enzyme Commission number data.

9. The method of claim 7, wherein the Enzyme Commission number feature comprises an Enzyme Commission class number and an Enzyme Commission sub-class number for the query molecule, wherein the method further comprises predicting an Enzyme Commission sub-sub-class number and an Enzyme Commission sub-sub-sub-class number for the query molecule based on similarity between query molecule structural features and the substrate structural features, wherein determining the microorganism taxon comprises determining the microorganism taxon based on the Enzyme Commission class number, the Enzyme Commission sub-class number, the Enzyme Commission sub-sub-class number, and the Enzyme Commission sub-sub-sub-class number.

10. The method of claim 1, wherein the machine learning model comprises a random forest model for predicting the enzyme feature associated with metabolism of the query molecule.

11. The method of claim 1, wherein generating the machine learning model comprises generating the machine learning model for predicting a plurality of enzyme features comprising the enzyme feature associated with the metabolism of the query molecule.

12. The method of claim 11, further comprising determining a plurality of microorganism taxa comprising the microorganism taxon associated with the metabolism of the query molecule based on the plurality of enzyme features predicted from the machine learning model.

13. The method of claim 1, wherein the query molecule comprises at least one of a vitamin-related molecule, an artificial sweetener-related molecule, and an alcohol-related molecule.

14. A system for metabolism-related prediction, the system comprising:
a data collection module for collecting:
protein data indicating a set of proteins associated with a set of microorganism taxa,
chemical reaction data associated with the set of proteins, and
substrate data comprising substrate structural features associated with a set of substrates associated with the set of proteins;
a metabolism module for predicting a protein feature associated with a metabolism of a query molecule, based on the protein data, the chemical reaction data, and the substrate data; and
a microorganism module for determining a microorganism taxon associated with the metabolism of the query molecule based on the protein feature predicted from the metabolism module for the query molecule.

15. The system of claim 14, further comprising a drug score module for predicting a drug score indicating a drug efficacy for a user for the query molecule based on the microorganism taxon and a microbiome characterization for the user.

16. The system of claim 15, further comprising a microbiome characterization module for determining the microbiome characterization based on a microorganism composition diversity dataset and a microorganism functional diversity dataset for the user.

17. The system of claim 15, further comprising a therapy module for determining a therapy for the user based on the drug score.

18. The system of claim 17, further comprising a therapy provision module for providing the therapy to the user.

19. The system of claim 14, further comprising a personalized dietary recommendation module for determining a personalized dietary recommendation for a user based on a microbiome characterization for the user and the microorganism taxon associated with the metabolism of the query molecule, and wherein the personalized dietary recommendation comprises at least one of a vitamin-related recommendation, an artificial sweetener-related recommendation, and an alcohol-related recommendation.

19. The system of claim 19, wherein the personalized dietary recommendation comprises the alcohol-related recommendation associated with the set of microorganism taxa comprising at least one of: Bacteroides uniformis (species); Holdemania filiformis (species); Turicibacter sanguinis (species); Eisenbergiella tayi (species); Erysipelatoclostridium ramosum (species); Dielma fastidiosa (species); Roseburia hominis (species); Catenibacterium mitsuokai (species); Solobacterium moorei (species); Eggerthia catenaformis (species); Allobaculum stercoricanis (species); and Lactobacillus (genus).

21. The system of claim 19, wherein the personalized dietary recommendation comprises the artificial sweetener-related recommendation associated with the set of microorganism taxa comprising at least one of: Enterobacteriaceae (family); Deltaproteobacteria (class); and Actinobacteria (phylum).

22. The system of claim 14, wherein the substrate structural features comprise at least one of a 3D structural feature, a product molecule feature, a drug feature.
